SEQUENCE LISTING (12 sequences)

SEQ ID NO: 1 - DNA, 808 bp, Mus sp.
agctcaaaccagcttaggctacacagagaaactatctaaaaaataattactaactacttaataggagattggatgttaagatctggtcactaagaggcagaattgagattcgaaccagtattttctacctggtatgttttaaattgcagtaaggatctaagtgtagatatataataataagattctattgatctctgcaacaacagagagtgttagatttgtttggaaaaaaatattatcagccaacatcttctaccatttcagtatagcacagagtacccacccatatctccccacccatcccccataccagactggttattgattttcatggtgactggcctgagaagattaaaaaaagtaatgctaccttattgggagtgtcccatggaccaagatagcaactgtcatagctaccgtcacactgctttgatcaagaagaccctttgaggaactgaaaacagaaccttaggcacatctgttgctttcgctcccatcctcctccaacagcctgggtggtgcactccacaccctttcaagtttccaaagcctcatacacctgctccctaccccagcacctggccaaggctgtatccagcactgggatgaaaatgataccccacctccatcttgtttgatattactctatctcaagccccaggttagtccccagtcccaatgcttttgcacagtcaaaactcaacttggaataatcagtatccttgaagagttctgatatggtcactgggcccatataccatgtaagacatgtggaaaagatgtttcatggggcccagacacgttctag

SEQ ID NO: 2 - DNA, 479 bp, Mus sp.
agcttttgtgtttgacccttccctgccaaaggcaactatttaaggaccctttaaaactcttgaaactactttagagtcattaagttatttaaccacttttaattactttaaaatgatgtcaattcccttttaactattaatttattttaaggggggaaaggctgctcataattctattgtttttcttggtaaagaactctcagtttctgtttttactacctctgtcacccaagagttggcatctcaacagaggggactttccgagaggccatctggcagttgcttaagatcagaagtgaagtctgccagttcctcccaggcaggtggcccagattacagttgacctgttctggtgtggctaaaaattgtcccatgtggttacaaaccattagaccagggtctgatgaattgctcagaatatttctggacacccaaatacagaccctggcttaaggcctgtccatacagtaggtttagct

SEQ ID NO: 3 - DNA, 960 bp, Homo sapiens
ttcctgaccactgagccttggtcctgattactgagccctggacccagtcaatgaccccctgttcctgatcactgagccctggaccaaatcactgagccctggaccagatcactgggccctggtcttgatcactgggccctggtcctgattactgagccctgatccctgatcactgggccctgttcctgaccactgagccctggaccagaccacggatccctgttcctgatcactgatctctggttcttttattatgcatattcattttgaaatctgattccttttctgagcatgtatcagtctgactagacactgagtcctgtctgatttctgagccttggccctcatgagtaagtgacctgcagtggtggagggaggcctccaggggagccgagaccctctcagtgcatgtactcactggtagatgaagaaatgaccccaatgacttgctccattcttccaggctcagaggggtgtgtaggccccaggaggacttggtggggagaagaccagcccaggccctgtgagctacacccagccccagcccctaaggggtcgccaggtctcgacttagcactggggagggggtacagtacaggagtggggacaggaaggtgaggggaggccatgccgtttgtattctcttgcttttctctctctcctgaagcctcttgaatagacctgcagaaatacccaaaatagccctgtggggtggctgagtcattgtgaacacagcccaggtcaggtgttccagccagagaactgctgttctgagaaacatgccccaaaaccgagacctggccaggtgtgcctggggcctgagcgaggggctgcagccacaggtaggcccagccccaaccagcccagagtcagctagggctttccaggtccagggttaggcagaggtcagccagggtcaaccacggtctatctgaggggagagacaagagacacagagac

SEQ ID NO: 4 - DNA, 1565 bp, Mus musculus
tgatagagaggagatgacagaaggaagagacacagatagacagaggtaggagatgtgcacctagagaaagggggcacaagcagagatgagagagagagagagagagagagagagagagagagagagagagagaggaagaagaagaaggagaaggagaaggagaaggagaaggagaaggagaaggagaagaagaaagaggagagacaaggtgttaatagagacagctcttatgttttgttagagatggagagacagtaactgagggacagactgagcaagaatcacactgagagagagagaaagaggcagaggctaaggctgggaaaggcagggttggccagagatggagccagggtcagacaccatcttgaaagtgccagtgacaagcaggaggtgacaggctggtacagtttctatcccacagtccacaggttccagtcacaggcctgtctccatgtggccacaggctagctccctgtctgccaagtctgtctgagaaggggtgggggagggggtctgggctgaggcaggccaagacttttccagtggagtggccagggactgggctgagggcatgatcactgcccaccattcccatggttctgggatacccaggatttggagcacacctacagccttcctgcctctctcacttccctggggtgttgagccacccatccttgcccatctcctgtcatgtcctcttggtgggtcagattccagcagtggtgataactgggaaacacggaattcaacatcatcaatagggtcatggacccccagtcccagtggcctatgctgggagtcccccatccccaaggctggtcagcctggccaggttggggtgaacctgcagctgcaggtgggggagtcactcatgctatttctggaaacaacctcagaaagtgagaaacatgtttttctgtatgaaacaaacatccatttgcatgtgcccttgtgagccaactctgcacagaatgaggtaactcggcagcgggggcgaggacatgttgggagtaagccttgtttctggtactgatatctgagcccccaacccccagattgtaaccctagcccatgtggaagaactacccttaggggtctattaactcaccacgctaggccatcatggagagccctagctgcctctaagcaggttccaccctaaacacgaaggaaaataatcaaggccatccattacccagaccactgggatggtgggcagggatcatgccctcagcccagtgcctggccactccactggaaaagtcctggcctgcctcagcccagcccccctcccccatccctccacagacaggcttggcaggcagggatagactgtggccacgtggagacaggcctgtgacaccagcctgtggactgtgggatagaaactgtaccagcctgtcacctccctggcttgtcactgcctcttccaagatggcgtctggtcctgtcccacaggctcaactctgtcccctctggctactcttgcctttcctagccttagcctctgtttgtctctgaatggtattctctctgctcagtttgtcaactgcagttgg

SEQ ID NO: 5 - DNA, 1680 bp, Homo sapiens
tattttaaagagtatgttctttcattatcctctgttattacttgtctacatttttattctgccaagaaggccgtggcaccgcgagctgtagacagagccgcggtctttctcgattgagtggctttggtggccatgccaccgcgctcttggggcagccgccttgccgctatgccgtggccaccctgtgtctgcccgattaatgctgccgtagccagctttcctgatgcacagtgatacaaataatgccactaagggaaagagaacagaaacgtaatgggcgctgagctgggaaaaccagggagaagactgatttattagagatttcagaaataaaattcacattcattatgatatctcattagtgaaaatttccattaggggattgtaaataatttaaagctttttttttttcagtgctatttaattatttcaatatcctctcatcaaatgtatttaaataacaaaagctcaaccaaaaagaaagaaatatgtaattctttcagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgataggactattttagggcctttttaaagctctattaaaactaacttacaacgattcaaaactgttttaaactatttcaaaatgattttagagccttttgaaaactcttttaaacactttttaaactctattaaaactaataagataacttgaaataattttcatgtcaaatacattaactgtttaatgtttaaatgccagatgaaaaatgtaaagctatcaagaattcacccagataggagtatcttcatagcatgtttttccctgcttattttccagtgatcacattattttgctaccatggttattttatacaattatctgaaaaaaattagttatgaagattaaaagagaagaaaatattaaacataagagattcagtctttcatgttgaactgcttggttaacagtgaagttagttttaaaaaaaaaaaaaactatttctgttatcagctgacttctccctatctgttgacttctcccagcaaaagattcttattttacattttaacctactgctctcccacccaacgggtggaatcccccagagggggatttccaagaggccacctggcagttgctgagggtcagaagtgaagctagccacttcctcttaggcaggtggccaagattacagttgacctctcctggtatggctgaaaattgctgcatatggttacaggccttgaggcctttgggagggcttagagagttgctggaacagtcagaaggtggaggggctgacaccacccaggcgcagaggcagggctcagggcctgctctgcagggaggtttta

SEQ ID NO: 6 - DNA, 3199 bp, Gallus gallus
gtgtgcaccccacccctgtcactatcccctctccccgctccccagggagattttgcagtggcccctgtaggagcttttagcacagcccccagcagcaagcaagcagaaagcactgctgtcgacagcttgtcagctgtgtgtgtttgctgaggaggatctgtcttttgctgaggccatcagtcttgtcctgctcaacctccatcgatgctggcccacctcaacacatctacccatctattccatctacaccaacatctccattcatcccacccacccaaacatgtccatccatcacaacacctccatccaacccgcacactccagacctccaatcattccatctacaccaccatgctgatctgcacagccactccaacgcaaccgtccattccatctacaccaatgtccatccatcccagccactccagcacctccagccatcccacccaccctatgtctccatccagccactggtggggtgcaggacatggggccagctctactgtaaggactggggtttttgcatggccccataccacttctgcagaagagacgcactgaaagtttggctgaccatttctccacggtagagttgtggcagttctgtaatttagggtcttttatccagtttggagatgggctgggatctcccagtccatggcaggcattcatgacactgggtttagtatctgatgggtgggatgtggctgaacttcattttctttccccagtgacaaagtttttgcagttgaatatgaattcctgctttctgctctagagttgtttttttcccaggacgtacacagggaatcagcagtcttcattctccctctgccatgtgtagactctctgccacacaggactgtgctgtcctcatgcccctgcgcccaaattgctgccctctgcccatgcctgccaagctgaccctgcaggctgccatgctggattgacatgagccctgagattggtacagaaatggtgattttggggttttctttgcactcaggaagctgaaggctcaatgctcagtgatggatttaccaaactgtccctgaggcagctgctcatgctggataaagtcactggagcacagggaaccaggcgctgggcagggatttctcatgggccccacttggaaagctgcaggctgcaaacctggctgccttcacgcctcaccctcatgaggacaacctcactaattattgattaaaagattttgctaaaccatctccagaagcaacaacccactgaggagcatgtgctgaattatacatcacagctccacggccctgcctcatagcaggctgcatggcacccacagtggcactcagagggaccacagggctgagacagccgggtctggtggtggggacacagctgagcataggatgagccccgggcagtgctgggctttgctaatgagcagaagtatggatagaaagcaaccccaggctacccagctgcagctcttgctctgtcgtgtcctttggtgaaactttaaacagtcgcctttttttttctctttcttttctggcttgccattaatttcaaacctgagagagacctaatttagtaaatgagatgcttcaggacacggctttaattagctgcagatggaggcaggcagtgctatcgtggggcctggatcgcacagggagctgatatcctcactagcagatacactcaggctgggtccctcccacattcatgccccagaccagagggtatatgctctgttccccacatcatcgctcccacactcttgcagccgttgagccccaacatcccaccagcacacggggctcagcacgcctggcgacatggcatcagcagagcaggccgcatggtacagctccatcacgacagctggggccacacaaagagctgggttactgtgggcagcaggctgaaacccgaaaacaagagctgggggctcagaatagccccgggagcagcaggggcctgggggtgagggtcaagcaccaggcccaggcgcgacacagcccttccaggaaggcacagcgctgtcagggtgcagcacgctcagccccaccatgcagctgtgaggccggggcatccccaagctaaatttacttctcagtctccaatcagaaactgaagctgaggggcccacgcgcgccaaaaaaaggaaacgaaacagtctccagaaagtccacctgacgttgtgaagcagagacgcgccgcgcaaaccggccgccatgtcacacacctcaggttggggcctttgcagactgagtctttgctgctgctgctcggggtgggtgcccacgcgctgggcacatgggatggggtacacacgtacacacacttgcacacccacaccccaaccacttcaggtgatgctggtgcagatgggtgccccccaggctgacccccccacgcatgggcctggccacactgctccatccgtgtctctgtccccatgtgccacccctgcccgctcccaccacgcgtcaacccaaatcctgagttaatcccacgactcctgcctgcttccagcgtccatggcagactggagatgccccaaaatgcagagcaggtttccctgaatctgagagatgaaatggagttatgggtgttcccctgcggcggaccgcaagtctgtaggaagctcagagccatcacacagcaattaaagaggaattaaattaaatcaataaatgttttaggcggctcagctgcagcaccacctgaccgaaacagcccgcttgcaaagaggagagcatttgcatggctgtggcaaaacagcaaccgcctgttgtgcagctgggatggtgttatctggaaagtgacgcgacccaggagggtaaacagctccaaactgagaccctgagcttgtcacaggttgtaacaggctgacataaacacctttagtttacgccgtggaaaaatattatcacctcaaatatagcaggttaataaaataaactcccaacggagcttcacacctgctttggaagggaagcagacacttgttttctgcttgatgttggctgtaggaagccatgtttcccgatgcaggagggccacaagatgcacactgacaacacaatgtgagctgagcttcgcccctgtttaagcccccaccacagggcttgtgcagcctagagcagacagggcgcaggggtggcaccgggctgggtgacatgggctggtctggggtgtctcactgagctct

SEQ ID NO: 7 - DNA, 4 bp, Homo sapiens
rgyw

SEQ ID NO: 8 - DNA, 4 bp, Homo sapiens
dgyw

SEQ ID NO: 9 - DNA, 15 bp, Artificial
  Synthetic
atgggatcggccatt

SEQ ID NO: 10 - DNA, 18 bp, Artificial
  Synthetic
atgggatagtcagccatt

SEQ ID NO: 11 - DNA, 21 bp, Artificial
  Synthetic
atgggatcgtacccttatgat

SEQ ID NO: 12 - DNA, 21 bp, Artificial
  Synthetic
atgggatagtacccttatgat

(19) United States
(12) Patent Application Publication
(10) Pub. No.: US 2006/0080745 A1
(43) Pub. Date: Apr. 13, 2006

(54) Gene reactivation by somatic hypermutation

(75) Inventors: P. Leif Bergsagel, Scottsdale, AZ (US); Davide F. Robbiani, New York, NY (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Cornell Research Foundation, Inc., Ithaca, NY (US)

(21) Appl. No.: 11/247,109

(22) Filed: Oct. 11, 2005

Related U.S. Application Data
(60) Provisional application No. 60/617,367, filed on Oct. 8, 2004.
(60) Provisional application No. 60/617,753, filed on Oct. 12, 2004.

(51) Int. Cl. A01K 67/027; C12N 5/06; C12N 15/87; C07K 16/18

(52) U.S. Cl. 800/14; 435/325; 435/326; 435/455; 530/387.1

(57) ABSTRACT

The invention provides an inactivated or attenuated gene functionally linked to a hotspot for somatic hypermutation. Inventive nucleic acids include inactivated genes operatively linked to immunoglobulin gene control elements. Embodiments of the invention include murine models of plasma cell disease and germinal center cell lymphoma.

### CROSS-REFERENCE TO RELATED APPLICATIONS

This patent application claims the benefit of U.S. Provisional Patent Application No. 60/617,367, filed Oct. 8, 2004, and U.S. Provisional Patent Application No. 60/617,753, filed Oct. 12, 2004.

### BACKGROUND OF THE INVENTION

Normal cells must reproduce, or replicate, their DNA with fidelity. When mistakes occur during replication, mutations are fixed in the progeny DNA. Cancer cells have higher mutation rates because their DNA repair systems have been disrupted. Accordingly, cells have multiple mechanisms that repair DNA so as to prevent mutations from being fixed in the genome. Cells not only control the rate of mutation, but also control the location in the genome at which mutations tend to occur. More important regions, such as those encoding proteins, have additional mechanisms of DNA repair (e.g., transcription coupled repair). When mutations occur in regions of the genome encoding proteins, the proteins produced can lose their function or have reduced half-lives. In addition, mutations occurring outside of the protein coding region can also affect protein expression. Nevertheless, there are rare situations where a higher rate of mutation is desirable. The antigen recognizing cells of the immune system are an example of cells in which a spatially-focused higher rate of mutation facilitates cellular function.

The immune system recognizes and reacts to foreign molecules or antigens. The antigen recognizing cells of the immune system comprise both T and B lymphocytes (or T and B cells). Antigens are recognized by antigen receptor molecules on the surface of lymphocytes. B cells recognize and bind to antigens through immunoglobulins (Igs) on their surfaces. Igs are multimeric glycoproteins made up of heavy and light chains. The Igs must have tremendous diversity to recognize all possible antigens. In order to generate a large repertoire of Ig proteins, the genes that encode the Ig heavy and light chains are organized into modular rearranging units. This allows for different cassettes of variable (V), joining (J), and diversity (D) segments to assemble in multiple combinations. However, Ig gene rearrangement alone does not create enough immunoglobulin diversity. Focused somatic “hypermutation” also contributes to the diversity in the Ig coding sequences and is required to produce Igs that recognize all antigens. Somatic hypermutation in the Ig genes is an ongoing process in B cell development which stimulates B cells that tightly bind to an available antigen to proliferate. Somatic hypermutation in the Ig gene is spatially controlled by the Ig gene enhancer elements and is accelerated by transcription.

The process of selecting B cells for expansion based on their ability to bind antigen occurs in the lymphoid organs in microscopic structures called germinal centers. Germinal centers are comprised of a variety of B cells in different stages of maturation. The ultimate functional utility of B cells in the immune response is to produce antibodies made up of Ig molecules that circulate, bind to, and inactivate antigens. Before B cells produce significant amounts of antibodies they must mature into plasma cells.

B cells can become cancerous producing leukemias and lymphomas. The most common type of lymphoma is germinal center B cell lymphoma. Plasma cells can also become neoplastic when the disease multiple myeloma develops. It is estimated that in 2003 there were 14,600 new cases of multiple myeloma and 10,900 deaths from multiple myeloma in the United States. In addition, plasma cells can produce excess antibody leading to other diseases such as amyloidosis and monoclonal gammopathies.

Because of the importance of germinal center B cell lymphoma and multiple myeloma it is desirable to develop animal models of these diseases. However, over expression in murine B cells of BCL-6, which is an oncogene commonly activated in lymphoma, results in premature B cell death rather than lymphoma. Similarly, animal mouse models using C-MYC, an oncogene involved in multiple myeloma, have produced animals with B cell neoplasms at an earlier stage of B cell differentiation rather than multiple myeloma. Therefore, the stage specific expression of oncogenes can facilitate the generation of animal models of B cell lymphoma and multiple myeloma. Moreover, the ability to activate a gene at specific points in a cell's development would be of great utility in the study of a wide range of biologic phenomena and could result in novel therapeutics.

The invention provides such nucleic acids, cells, animals, and methods. These and other advantages of the invention, as well as additional inventive features, will be apparent from the description of the invention provided herein.

### BRIEF SUMMARY OF THE INVENTION

The invention provides an isolated and purified nucleic acid comprising an inactivated or attenuated gene functionally linked to a hotspot for somatic hypermutation. The gene of the nucleic acid is inactivated by a sequence that interferes with gene expression. The sequence inactivating the gene can be any suitable sequence. Examples of suitable sequences comprise an upstream start codon (in or outside of a Kozak consensus sequence), an upstream open reading frame, a stem-loop structure, a repressor binding sequence (e.g., an iron responsive element), a premature stop codon, a frame shift mutation, a mutant or non-mutant splice donor site, a mutant or non-mutant splice acceptor site, an internal ribosome entry site, a sequence that accelerates RNA degradation, or a sequence that encodes amino acids that accelerate protein degradation, or any other sequence intended to inactivate or attenuate expression. The nucleic acid comprising the inactivated or attenuated gene desirably is operatively linked to one or more immunoglobulin gene regulatory elements. These Ig gene regulatory elements cause somatic hypermutation in the inactivated gene. Such hypermutation disables the sequence inactivating the gene, thereby causing its derepression.

The nucleic acid sequence inactivating the gene preferably overlaps with, or is within about one to about five nucleotides of, a “rgyw” or a “dgyw” nucleic acid sequence, where “r” is a purine (i.e., guanine or adenine), “g” is guanine, “y” is a pyrimidine (i.e., thymine, uracil, or cytosine), “w” is adenine, thymine, or uracil, and “d” is any nucleotide except cytosine (i.e., adenine, guanine, thymine, or uracil). These tetranucleotides and related codons are not essential, but are commonly found in hotspots for somatic hypermutation.

The invention also provides embryonic stem cells transformed with any of the nucleic acids disclosed herein comprising an inactivated or attenuated gene functionally linked to a hotspot for somatic hypermutation. The invention also provides methods of generating transformed animal cells, preferably of the hematopoietic lineage, transgenic animals, chimeric animals, transgenic or chimeric blastocysts, and their progeny containing nucleic acids engineered to contain an inactivated gene functionally linked to a hotspot for somatic hypermutation. The invention additionally provides cells and antibodies isolated from such animals, related monoclonal cell lines and hybridomas, and related methods of regulating gene expression, producing antibodies, and the like. The invention has many uses, including without limitation, the study of plasma cell diseases and germinal center cell lymphoma.

### BRIEF DESCRIPTION OF THE DRAWINGS

FIG. 1 is the DNA sequence of a murine immunoglobulin kappa 3′enhancer (SEQ ID NO: 1).

FIG. 2 is the DNA sequence of a lymphocyte-specific enhancer in the mouse immunoglobulin kappa (SEQ ID NO: 2).

FIG. 3 is the DNA sequence of a human Ig C alpha 3′ enhancer gene intron (SEQ ID NO: 3).

FIG. 4 is the DNA sequence of the mouse immunoglobulin heavy-chain locus enhancer which cross hybridizes with the rat 3′ enhancer element (SEQ ID NO: 4).

FIG. 5 is the DNA sequence of the intron of the germline human kappa immunoglobulin gene connecting the J and C regions (SEQ ID NO: 5).

FIG. 6 is the DNA sequence of the chicken Ig lambda light chain gene, silencer, enhancer, and matrix associated region (SEQ ID NO: 6).

FIG. 7 is a diagram illustrating second generation engineered oncogene constructs activated by somatic hypermutation.

FIG. 8 is a diagram illustrating a construct used to generate the transgenic Vk*HAMYC mouse.

FIG. 9A is a graph illustrating the percentage of transgenic mice with monoclonal spikes detected in serum by protein electrophoresis at the indicated weeks of age. Squares indicate transgenic mice, triangles indicate wild type mice, and n is the number of mice for each condition.

FIG. 9B is a protein electrophoresis gel of serum isolated from Vk*HAMYC transgenic (tg) and wild-type (wt) mice demonstrating a monoclonal antibody spike (pointed by triangle) increasing in intensity with age. “A” represents albumin, β is the beta fraction of serum, and γ is the gamma fraction of serum containing immunoglobulins.

FIG. 9C is a serum protein electrophoresis gel of serum isolated from Vk*HAMYC (tg) and wild-type (wt) mice demonstrating multiple monoclonal antibody spikes in a portion of older transgenic mice.

FIG. 9D is an image an immunofixation experiment which detected an IgG1 monoclonal antibody spike in Vk*HAMYC mice. The triangle points to a monoclonal spike in serum protein electrophoresis (left lane). Right lanes are different dilutions of the same mouse serum (1:5, 1:10, 1:50) reacting with IgG1 antiserum.

FIG. 10A is a protein electrophoresis gel of serum isolated from NP-CGG-immunized mice. Serum was obtained 1 day before (pre) and 2 weeks after (post) NP-CGG immunization. Representative serum protein electrophoresis traces are shown for a representative wild type mouse (wt) and a responsive transgenic mouse (tg). The filled arrowhead points to a monoclonal antibody spike in the serum of an NP-immunized transgenic mouse.

FIG. 10B is a protein electrophoresis gel of serum isolated from NP-CGG-immunized mice. The filled arrowheads in the top panel point to monoclonal antibody spikes in sera of NP-immunized transgenic mice. Antibody spikes in non-immunized transgenic controls are indicated by empty arrowheads. The bottom panel shows NP-reactivity detected in sera of NP-immunized transgenic mice (arrows), but not in non-immunized transgenic controls.

FIG. 11 is a diagram illustrating the construct used to generate a transgenic BCL-6 mouse.

### DETAILED DESCRIPTION OF THE INVENTION

The invention provides an isolated and purified nucleic acid comprising an attenuated or inactivated gene functionally linked to a hotspot for somatic hypermutation, in which the gene is inactivated by a nucleic acid sequence that impedes or blocks gene function by impeding or blocking gene expression. The term “hotspot” as used herein, refers to a region of a nucleic acid sequence in which mutations are concentrated, as compared to the entire nucleic acid sequence (see, e.g., Rogozin et al., Pac. Symp. Biocomput., pp. 409-20 (2005)). The impediment to gene expression is preferably not at the level of transcription (i.e., copying of the DNA sequence into an RNA sequence). Rather, the impediment is preferably at the level of RNA stability, RNA processing, protein translation, protein trafficking, or protein stability. It is also possible to impair protein function. Additionally, the block is caused by a small change or addition to the sequence of the nucleic acid such that the repression of gene activity can be disabled by a single or few mutations.

The functional linkage of the inactivated gene to a hotspot for somatic hypermutation causes an increased rate of mutation within the attenuated or inactivated gene when in a suitable host cell. This preferably causes temporally-regulated elimination (i.e., derepression) of the inactivating sequences in the gene, which can be driven by the process of somatic hypermutation. Thus, the invention provides stage-specific gene expression that can be stimulated by host cellular mutation rates.

The inactivated or attenuated gene preferably encodes a protein or RNA with a normal or desirable cellular function, rather than one that merely acts as a reporter. An inactivated gene is a gene which is not expressed until a mutation activates the gene and an RNA or protein is produced. An attenuated gene is one which is expressed in a host cell, but is not expressed at levels sufficient to cause the desired effect or is expressed at such low levels that the protein or RNA cannot perform its normal cellular function.

The attenuation can be caused by any suitable mechanism. For example, attenuation can result from (without limitation) reducing the level of protein or RNA synthesis, disrupting the protein's normal binding or biochemical activity, or accelerating the protein's degradation. Similarly, inactivation can result in, for example, insufficient levels of cell surface receptors, receptors that do not bind their ligands, inadequate intracellular signal transduction, weak cellular proliferation, unprotected programmed cell death, a lack of transcriptional activation, or absent DNA recombination.

The nucleic acid sequence attenuating or inactivating the gene can be of any suitable structure. For example, the nucleic acid sequence inactivating the gene can be one or more start codons, a Kozak sequence, or an open reading frame upstream of the inactivated gene's normal start codon such that translation will initiate at these artificial points and compete with the translation of the normal open reading frame from the same mRNAs. Similarly, multiple start codons can be cloned upstream of the gene's normal start codon such that all three reading frames can be translated from the same mRNA. In this way the efficiency of the non-productive upstream reading frames will out-compete the legitimate protein encoding open reading frame downstream. In addition, the short artificial upstream open reading frames are likely to be only transiently translated leading to more rapid mRNA degradation. Similarly, the attenuating or inactivating sequences that can comprise internal ribosome entry sites that disrupt normal translation by ribosomes entering downstream from the normal start codon. The placement of such internal ribosome entry sites in intronic sequences will facilitate this function. These mechanisms will result in a reduction in the translation of the normal downstream open reading frame.

The invention also includes inactivating stem-loop structures and structures, such as iron responsive elements, which when present in mRNA can inhibit translation by making the normal start codon inaccessible for ribosome entry. In addition, the invention can include a nucleic acid sequence bound by a transcriptional repressor protein that acts to reduce transcription of the inactivated gene. Furthermore, the inventive nucleic acids include those with premature stop codons or a frame shift mutation, preferably near the beginning of the normal open reading frame, that inactivate the gene by producing truncated or nonfunctional proteins. Such mutations can also increase the degradation of the mRNA. Alternatively, the inventive nucleic acids can contain a mutant splice donor site, a mutant splice acceptor site, or a sequence that accelerates RNA degradation so that the level of functional mRNA is inadequate to produce sufficient protein. Another attenuating or inactivating sequence is one that encodes amino acids that accelerate protein degradation, such as a target sequence for ubiquitinization, and result in insufficient protein levels for cellular function. Similarly, the gene can be inactivated by the use of one or more splice donor and/or acceptor sites which can cause a portion of the gene product necessary for function to be spliced out of the mature RNA or mRNA. Preferably the attenuating or inactivating sequence can be disabled by substitution, deletion, or addition of 1, 2, 3, 4, 5, 5-10, or 1-15 nucleotides, which disables the inactivating sequence to derepress the gene.

Expression of the gene can be driven by any suitable promoter. For example, the gene can comprise its own natural promoter, or a transcriptionally active portion thereof, or it can comprise a heterologous promoter. The heterologous promoter can be one selected from the species of the host cell to be transduced, or it can be from another organism or virus. For example, the promoter can be a viral LTR. Additionally, the promoter can be a weak, intermediate, or strong promoter. This promoter is preferably a promoter that drives the expression of an immunoglobulin. When a promoter of an immunoglobulin is employed, the attenuated or inactivated gene preferably does not encode an immunoglobulin or portion thereof. The mouse Vk21E promoter and non-murine homologs thereof are among the preferred Ig promoters useful in the context of the invention. While not desiring to be bound by any particular theory, it is believed that the somatic mutation rate will generally correlate with the level of transcription.

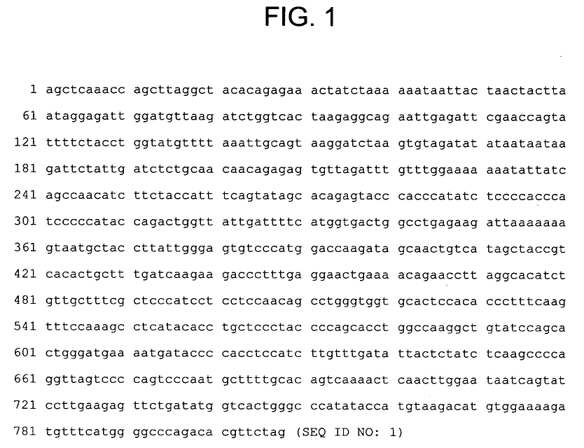

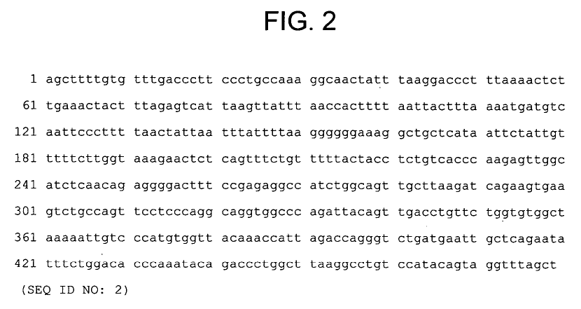

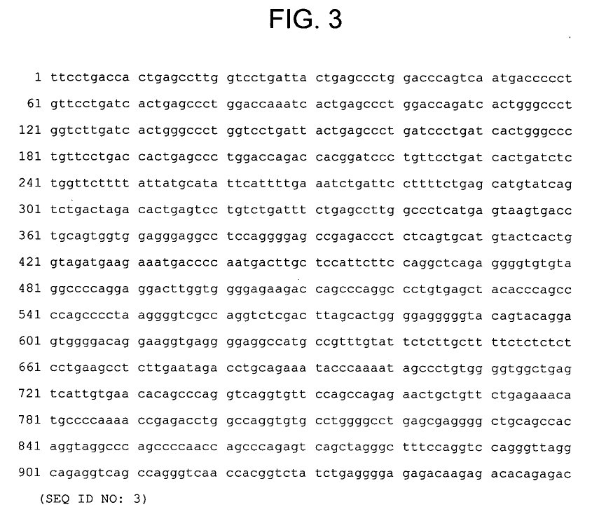

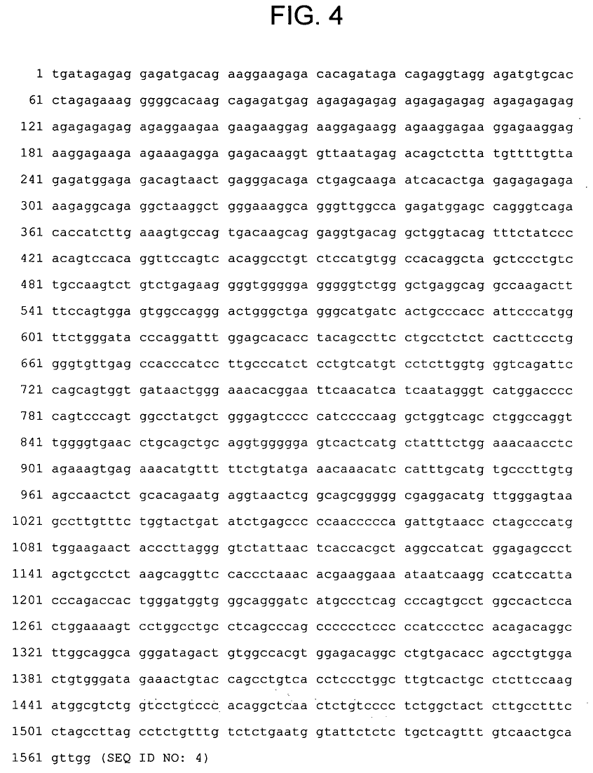

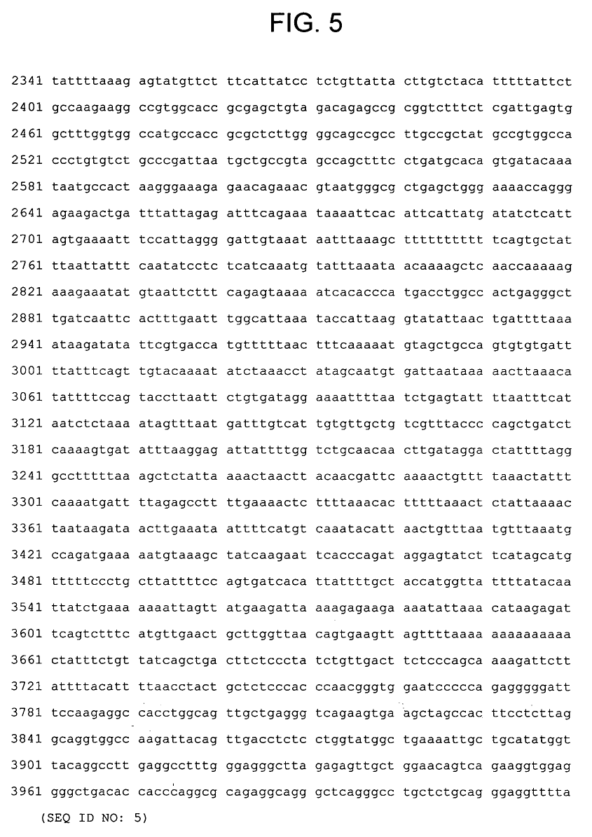

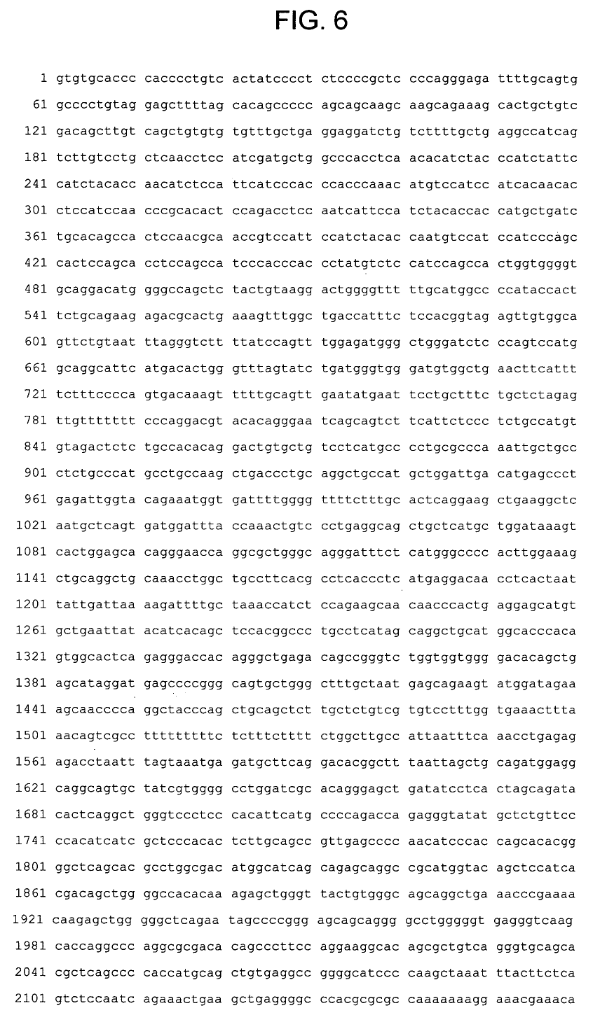

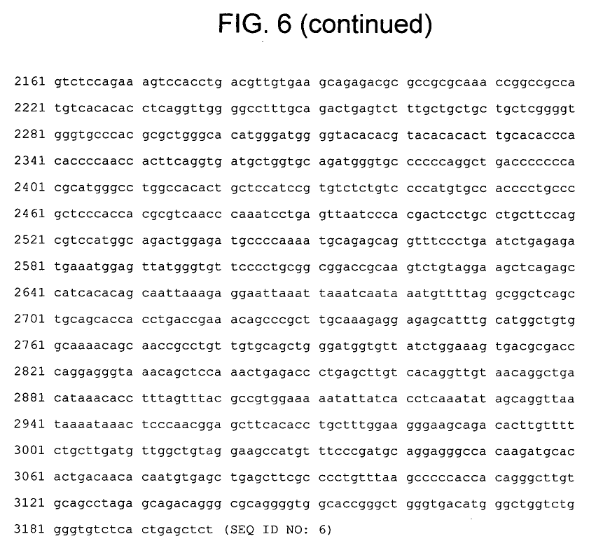

The nucleic acid encoding the inactivated gene also comprises an immunoglobulin transcription enhancer, which increases the rate of somatic mutation. FIGS. 1-6 present exemplary immunoglobulin enhancer sequences (SEQ ID NOs: 1-6). The Ig enhancer comprises two functional portions. First, the Ig enhancer comprises an “intronic enhancer,” which intronic enhancer is preferably placed in an intronic sequence of the attenuated or inactivated gene, and is preferably within 3 kb, more preferably 2 kb, and yet more preferably 1 kb of the start site for transcription from the promoter driving the expression of the attenuated or inactivated gene. The intronic Ig enhancer also comprises an MAR (matrix attachment region) region. The Ig enhancer also comprises a 3′ enhancer. The MAR region is preferably separated from the intronic enhancer region and preferably is 3′ of at least one exon of the gene and 5′ of the polyadenylation signal. Other suitable embodiments, however, e.g., wherein the gene comprises a single exon, are contemplated herein. Additionally, the sequence which attenuates or inactivates the gene is preferably disarmed by a mutation which can occur within 2 kb, preferably 1 kb, more preferably about 500 base pairs of the start site of transcription.

The inactivated gene construct is, therefore, preferably designed such that the inactivated or attenuated gene is spliced following transcription (i.e., the gene comprises an intron) and an Ig intronic enhancer, such as the kappa intronic enhancer, is cloned in the intronic sequences of the inventive nucleic acid. The 3′ Ig enhancer is preferably 3′ of the inactivated gene. Although any 3′ Ig enhancer is adequate, the murine kappa gene 3′ enhancer is among the preferred embodiments at this site. The orientation of the 3′ Ig enhancer is not vital, i.e., it can be inserted in either direction. The distance between the start point of transcription and the 3′enhancer can be about 4 kb or less, e.g., about 3 kb or less, or even about 2 kb or less. More preferably, the distance between the start point of transcription and the 3′ enhancer is about 20 kb or less, e.g., about 18 kb or less, or even about 15 kb or less.

Both the intronic enhancer and the 3′ enhancer include one or more E-box sequences (caggtg), which are present in all Ig enhancers and whose presence has been correlated with somatic hypermutation independent of transcriptional activity. Furthermore, PU.1 and EM5 binding sequences are preferably incorporated into the 3′ enhancers for optimal somatic hypermutation targeting to rgyw (SEQ ID NO: 7) nucleic acid sequences. The PU.1 sequence is gaggaa, and the EM5 sequence is gaaaa. These sequences are preferably unaltered in the Ig enhancers incorporated into the inventive nucleic acids.

The constructs (including a suitable promoter, intronic immunoglobulin gene enhancer and MAR, and 3′ immunoglobulin gene enhancer) will produce hypermutation rates with a 5′ boundary at about the start of transcription and extending downstream for about 2 kb. Thus, it is desirable to clone the sequences that inactivate the expressed gene within about 2 kb of the transcriptional start site, more preferable within about 1 kb of the transcriptional start site, most preferably within about 500 base pairs of the transcriptional start site.

The nucleic acid sequence inactivating the gene preferably overlaps with or is within about one to about five nucleotides of a rgyw (SEQ ID NO: 7) or dgyw (SEQ ID NO: 8) nucleic acid sequence. These rgyw and dgyw motifs have been identified as preferred targets for the B cell's hypermutation machinery. In particular, agc- and agt-triplets can be desirable as intrinsic hot-spots of the hypermutation machinery. Not all tetramers with an rgyw or dgyw consensus or all agc/t codons are targeted equally by the hypermutator. Accordingly, the inclusion of these tetramers and codons in the inventive nucleic acid is optional.

The process of somatic hypermutation will alter the sequence of the inventive nucleic acid in a suitable cellular setting, which is preferably a cell in the B cell lineage, most preferably a germinal center B cell. This process can be focused by rgyw and dgyw nucleic acid sequences but also will occur throughout the cloned sequence from the transcriptional initiation site downstream to about 2 kb from the transcriptional initiation site. Somatic hypermutation will result in mutation of the inactivating sequence because of the high rate of mutation achieved in the suitable cellular setting and because a large number of cells are undergoing somatic hypermutation focused in the inventive nucleic acid. Mutations including point mutations, deletions, and insertion mutations of about 1 to about 5 nucleotides can reverse the inactivating effect of the inactivating or attenuating sequence which yields revertant cells. The revertants will express adequate amounts of functional protein which result in a useful phenotype. The phenotype preferably can be selected for (naturally or experimentally) in appropriate biological systems. For example, without limitation, an inactivated oncogene can be activated by somatic hypermutation leading to hyperproliferation of the revertant cells.

The inventive combination of promoters, Ig enhancers (both intronic and 3′ of the coding sequence), and optionally rgyw or dgyw nucleic acid sequences within about 1 to about 5 bases of the inactivating sequence, preferably produces somatic hypermutation such that there is a mutation rate of at least about 10⁻⁵ mutations per base pair per generation, preferably at least about 10⁻⁴ mutations per base pair per generation, and more preferably at least about 10⁻³ mutations per base pair per generation in the inactivated gene. Such mutations include point mutations, insertions of about 1 to about 5 bases, and deletions of about 1 to about 5 bases.

The inventive nucleic acids can be inserted into any suitable cell. Preferably, the transformed cell is either a B cell or a cell which can propagate and/or mature into a B cell. More preferably, the B cell is a germinal center B cell.

The inventive nucleic acids include, without limitation, those where the inactivated gene is selected from the group consisting of c-MYC, BCL-1, BCL-2, BCL-3, BCL-6, N-MYC, L-MYC, v-MYC, MMSET, MAF, FGFR-3, MUM1/IRF-4, ras family members, viral receptor genes, and site specific recombinases. Suitable ras family members include RAN, H-RAS, and K-RAS. Inventive viral receptors include proteins such as the avian retroviral receptor, TVA, which has been expressed under a variety of mammalian promoters in transgenic mice, thus rendering mice susceptible to infection with avian leukosis virus-derived gene vectors. Suitable site-specific recombinase genes include the flp and Cre recombinases, which have been used to generate lineage specific knock-outs of a variety of genes.

Inventive compositions include embryonic stem cells (ES cells) transformed with nucleic acids encoding an inactivated gene functionally linked to a hotspot for somatic hypermutation. Such inventive ES cells can be derived from, for example, mouse, rat, pig, or human embryos. The ES cells can be maintained in a totipotent state by culture in Leukemia Inhibitory Factor with or without a feeder layer of supporting cells. Inventive ES cells can be transformed before or after the initiation of differentiation towards, for example, blood, muscle, nerve, or vascular cells. The inventive transformed ES cells can also be reinjected into blastocysts for development into chimeric animals and potential population of the germline.

The invention also provides a method of transforming animal cells with nucleic acids encoding an inactivated gene functionally linked to a hotspot for somatic hypermutation. Such animal cells can include avian cells, such as chicken cells, and preferably are avian blood cells. Alternatively, such animal cells can include mammalian cells, preferably cells of the hematopoietic lineage, more preferably cells of the B lymphoid lineage. The hematopoietic lineage cells optionally can be identified by the expression of cell surface antigens such as, for example, CD34, CD3, CD19, CD10, CD20, or CD15. Similarly, the B cell lineage can be defined by the expression of, for example, CD19, CD10, the rearrangement of Ig genes, or the expression of cytoplasmic or surface Ig.

Furthermore, the invention provides transgenic animals and their progeny whose germ cells and somatic cells have a nucleic acid comprising an inactivated gene functionally linked to a hotspot for somatic hypermutation. Such transgenic animals can be, for example but without limitation, transgenic zebra fish, mice, rats, or pigs.

Embryonal cells at various developmental stages can be used to introduce transgenes for the production of transgenic animals. Different methods are used depending on the stage of development of the embryonal cell. The zygote is the preferred target for micro-injection. In the mouse, for example, the male pronucleus reaches the size of approximately 20 micrometers in diameter, which allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. (USA), 82: 4438-4442 (1985)). As a consequence, all cells of the transgenic non-human animal will carry the incorporated transgene. This effect will, in general, also be reflected in the efficient transmission of the transgene to offspring of the founder since 50% of the germ cells will harbor the transgene. Micro-injection of zygotes is the preferred method for incorporating transgenes. U.S. Pat. No. 4,873,191 describes a method for the micro-injection of zygotes.

Retroviral infection can also be used to introduce transgenes into a non-human animal. The developing non-human embryo can be cultured in vitro to the blastocyst stage. During this time, the blastomeres can be targets for retroviral infection (Janenich, Proc. Natl. Acad. Sci. (USA), 73: 1260-1264 (1976)). Efficient infection of the blastomeres is obtained by enzymatic treatment to remove the zona pellucida (Hogan et al., in Manipulating the Mouse Embryo, COLD SPRING HARBOR LABORATORY PRESS, Cold Spring Harbor, N.Y. (1986)). The viral vector system used to introduce the transgene can be a replication-defective retrovirus carrying the transgene (Jahner et al., Proc. Natl. Acad. Sci. (USA), 82: 6927-693 (1985)). Transfection is easily and efficiently obtained by culturing the blastomeres on a monolayer of virus-producing cells (Van der Putten, supra; Stewart et al., EMBO J, 6: 383-388 (1987)). Alternatively, infection can be performed at a later stage. Virus or virus-producing cells can be injected into the blastocoele (Jahner et al., Nature, 298: 623-628 (1982)). Most of the founders will be mosaic for the transgene since incorporation occurs only in a subset of cells which form the transgenic animal. Further, the founder can contain various retroviral insertions of the transgene at different positions in the genome which generally will segregate in the offspring.

Similarly, the invention provides transgenic blastocysts, chimeric animals, and progeny thereof, a portion of whose cells comprise a nucleic acid comprising an inactivated gene functionally linked to a hotspot for somatic hypermutation. Such chimeras can be, for example, of murine, rat, or porcine origin. Such chimeric animals can be generated by, for example the injection of transformed ES cells into blastocysts or the aggregation of ES cells with morula-stage embryos.

In addition, it is also possible to introduce transgenes into the germline, albeit with low efficiency, by intrauterine retroviral infection of the mid-gestation embryo (Jahner et al., supra (1982)). Additional means of using retroviruses or retroviral vectors to create transgenic animals known to the art involve the micro-injection of retroviral particles or mitomycin C-treated cells producing retrovirus into the perivitelline space of fertilized eggs or early embryos (International Patent Application Publication WO 90/08832, and Haskell and Bowen, Mol. Reprod. Dev., 40: 386 (1995)).

Another type of target cell for transgene introduction is the ES cell. ES cells are obtained by culturing pre-implantation embryos in vitro under appropriate conditions (Evans et al., Nature, 292: 154-156 (1981); Bradley et al., Nature, 309: 255-258 (1984); Gossler et al., Proc. Acad. Sci. (USA), 83: 9065-9069 (1986); and Robertson et al., Nature, 322: 445-448 (1986)). Transgenes can be efficiently introduced into the ES cells by DNA transfection by a variety of methods known to the art, including, for example, calcium phosphate co-precipitation, protoplast or spheroplast fusion, and lipofectin and DEAE-dextran-mediated transfection. Transgenes can also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, see Jaenisch, Science, 240: 1468-1474 (1988)).

The most important advantage of ES cells for gene transfer into animals is that ES cells carrying the transgene can be selected for before being injected into a blastocyst. For example, ES cells were infected with retroviral vectors, or transfected with plasmids, carrying the neo gene. This gene confers resistance to the antibiotic G418. Only ES cells that have taken up the neo gene grow in medium containing G418, and these G418-resistant cells can be introduced into mouse blastocysts. Not only can the resulting animals have neo integrated into their genomes, as shown by Southern blotting, but also the gene can be transmitted to the offspring, and cell lines from the F2 generation are G418-resistant. Alternatively, the polymerase chain reaction can be used to screen for ES cells which have integrated the transgene. This technique obviates the need for growth of the transfected ES cells under appropriate selective conditions prior to transfer into the blastocoel because ES cells can be manipulated in vitro before injection into the embryo, homologous recombination can be used to produce transgenic animals with mutations, specific genes or to replace a mutant gene with the normal equivalent.

One skilled in the art will recognize that progeny carrying the transgene can be identified by Southern blot analysis, by polymerase chain reaction (PCR), and/or by Northern blot analysis.

Once the transgenic or chimeric animals have been produced and identified, the animals, or their progeny, can be allowed to grow until tumors resulting from the expression of the activated gene are produced. One skilled in the art recognizes that aberrant eating patterns, loss of appetite, lethargy, aberrant growth patterns, and weight loss are external indicators that tumors are developing within the transgenic animal.

Animals having an abnormality, such as tumor growth, then can be used to harvest various differentiated cells, such as without limitation, plasma cells, lymphoid cells, monocytes, macrophages, B-cells, T-cells, neutrophils, erythrocytes, eosinophils, platelets, and the like. Additionally, the animal can be sacrificed, and differentiated cells populating the peripheral blood organs, spleen, lymph nodes, tonsils, blood, bone marrow, or other tissue(s) can be cultured. Preferred cells are lymphoid cells, typically plasma cells or germinal center cells

The invention also provides a method of studying plasma cell diseases including, but not limited to, multiple myeloma, plasmacytoma, plasma cell leukemia, monoclonal gammopathies, cytoglobulinemias, and amyloidosis. Similarly, the invention provides methods of studying germinal center cell lymphomas, for example, but not limited to, follicular lymphomas, small cleaved cell lymphomas, and diffuse large cell lymphomas. The inventive methods include the use of transformed animal cells, transgenic animals, chimeric animals, blastocysts, ES cells, and their progeny after transformation with a nucleic acid comprising an inactivated gene functionally linked to a hotspot for somatic hypermutation. Such cells, blastocysts, and animals optionally can be, for example, human, mouse, rat, or zebrafish origin. The methods of studying plasma cell diseases preferably employ an inactivated oncogene known to be involved in plasma cell neoplasms such as without limitation, c-MYC, MMSET, FGFR-3, or BCL-1. Conversely, the methods of studying germinal center cell lymphomas preferably employ an inactivated gene that is a gene known to be involved in such lymphomas. For example, without limitation, the genes BCL-2, BCL-6, or p53 are useful in studying germinal center cell lymphoma.

A preferred embodiment of the invention for studying plasma cell disease is the generation of mice transgenic for a stop-inactivated c-MYC oncogene (e.g., a c-MYC oncogene inactivated by a premature stop codon), which is engineered in a way to be turned on sporadically by somatic hypermutation that accompanies normal B cell maturation. In this embodiment, the stop-inactivated c-MYC transgenic or chimeric mice comprising the inventive host cells spontaneously develop a phenotype resembling human monoclonal gammopathy of uncertain significance and multiple myeloma, with clonal plasma cell expansions in the bone marrow.

Furthermore, a preferred embodiment of the invention for studying germinal center cell lymphoma uses mice transgenic for stop-inactivated BCL-6 oncogene, engineered in a way to be turned on sporadically by somatic hypermutation that accompanies normal B cell maturation. In this embodiment, the stop-inactivated BCL-6 mice spontaneously develop a splenic white pulp lymphoma similar to human germinal center B cell lymphoma. The foregoing effect is in marked distinction from transgenic mice having a normal BCL-6 oncogene controlled by the same promoter, which have a block in B cell development at the pro-B to pre-B cell stage.

The inventive nucleic acids described can be inserted into any suitable vector. Suitable vectors include, without limitation, viral vectors. Suitable viral vectors include, without limitation, retroviral vectors, alphaviral vectors, vaccinial vectors, adenoviral vectors, adeno associated viral vectors, herpes viral vectors, and fowl pox viral vectors, and preferably have a native or engineered capacity to transform target cells. Additionally, the vectors useful in the context of the invention can be “naked” nucleic acid vectors (i.e., vectors having little or no proteins, sugars, and/or lipids encapsulating them), or can be complexed with other molecules. Other molecules that can be suitably combined with the inventive nucleic acids include, without limitation, viral coats, cationic lipids, liposomes, polyamines, gold particles, and targeting moieties such as ligands, receptors, or antibodies that target cellular molecules.

The invention also provides a method of making monoclonal antibodies, cell lines, and hybridomas producing monoclonal antibodies. Methods of producing monoclonal antibodies are well understood in the art (Harlow et al., ANTIBODIES: A LABORATORY MANUAL, Cold Spring Harbor Laboratory (1988)). In this inventive embodiment, an antibody-producing animal comprising a nucleic acid of the invention is immunized with an antigen of interest, which causes the production of plasma cells specific for the antigen. While not desiring to be bound by any particular theory, it is believed that the B cells that react with the antigen undergo an increased rate of somatic hypermutation. This effect encourages the inactivated or attenuated gene to become activated. If the attenuated or inactivated gene is an oncogene, increased proliferation and higher cell counts of the reactive B cells can be found in the animal's lymphoid organs and blood. Thus, the immunized animal produces large amounts of antibody specific to the antigen of interest. Preferably, the animal is immunized with the antigen of interest before six months of age. The animal optionally can be repeatedly re-immunized with the antigen of interest. Antibody to the antigen of interest can be harvested from the serum of immunized animals by techniques established in the art (Harlow et al., ANTIBODIES: A LABORATORY MANUAL, Cold Spring Harbor Laboratory (1988)). Antibody producing animals can include, without limitation, mice, rats, sheep, and chickens.

In addition, in vivo monoclonal antibody production can be accelerated by immunizing the progeny of a cross between the transgenic animals with an attenuated or inactivated oncogene operatively linked to a hotspot for somatic hypermutation, preferably with an inactivated c-MYC, with animals deficient in tumor suppressor genes, e.g., the p16 tumor suppressor gene (see e.g., Serrano et al., Cell, 85(1): 27-37 (1996)). The progeny of such a cross will more readily produce monoclonal antibodies after immunization with the antigen of interest.

Antibody producing cells from the inventive animals can be isolated and optionally further adapted for in vitro cell culture. Methods of adapting animal cells to in vitro culture are well understood in the art (Pollard et al., METHODS IN MOLECULAR BIOLOGY VOL. 5: ANIMAL CELL CULTURE, Humana Press (1990); Masters, ANIMAL CELL CULTURE—A PRACTICAL APPROACH, 3d ed., Oxford University Press (2000)). Similarly, the harvesting of antibodies from cell culture supernatant is readily accomplished by the skilled artisan (Harlow et al., ANTIBODIES: A LABORATORY MANUAL, Cold Spring Harbor Laboratory (1988)).

The invention also provides hybridomas that produce the monoclonal antibodies described herein. Methods for generating hybridomas are well known in the art (see, e.g., Harlow et al., supra). In this respect, immunized transgenic animals will have more cells reactive with the antigen available for fusion with a suitable fusion partner, or the reactive cells can be isolated or used (or both) alone. The fusion partner is preferably an established myeloma cell line, which is also preferably resistant to HAT medium. In a more preferred embodiment, the oncogene is a c-MYC oncogene. Such fusions and their generation are well known within the art (Harlow et al., supra).

The following examples further illustrate the invention but, of course, should not be construed as in any way limiting its scope.

### EXAMPLE 1

This example demonstrates that an inactivated gene can be cloned in a hotspot of somatic hypermutation such that it is activated by somatic mutation. This example also illustrates an in vivo system that allows the determination of the effects of turning on oncogenes by somatic hypermutation (SH), which, here, is an inactivated c-MYC gene operatively linked to immunoglobulin kappa control elements in a transgenic mouse. During B cell development, the c-MYC gene was activated and resulted in a plasma cell neoplasm in the affected mice.

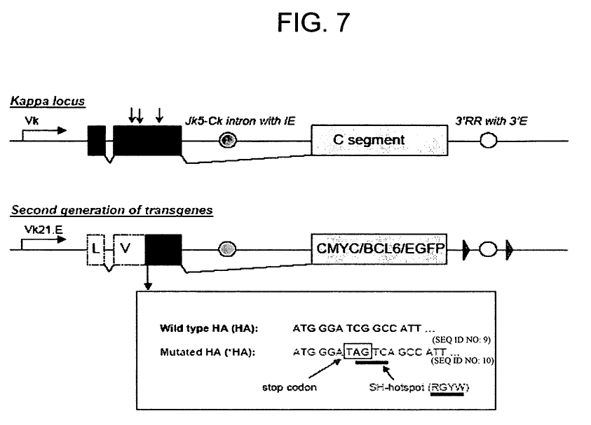

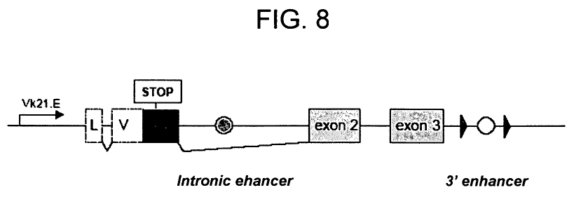

This embodiment of the invention is demonstrated in FIG. 7 which shows the normal Ig kappa light chain and exemplary inventive constructs with a premature stop codon near the transcriptional start site. Specifically for the c-MYC transgenic mice, C57BL/6 mice (Vk*HAMYC mice) were engineered to express a stop-mutated, HA-tagged (SEQ ID NO: 9 and SEQ ID NO: 10), human c-MYC oncogene in B cells, under the control of mouse kappa light chain regulatory elements (FIG. 8). Ig kappa gene promoter and enhancer elements were chosen and positioned to drive SH, and the stop-mutation was overlapped with an rgyw hotspot (FIG. 8). Thus, the transgenic message should be constitutively expressed, but not translated, unless the cell entered a germinal center reaction, where SH was able to sporadically revert the stop codon and allow c-MYC translation. Transfection of stop-mutated and non-mutated c-MYC constructs into 293T cells verified the absence of translation in the presence of the stop codon. Conversely, transfection of constitutively hypermutating Ramos cells with a stop-mutated enhanced green fluorescent protein (EGFP) plasmid verified hypermutability of such constructs

Two independent transgenic lines were generated. Transgenic splenocytes showed proper induction and achieved high levels of transgenic message expression upon lipopolysaccharide (LPS) stimulation, as is required for SH to occur at the transgenic locus. Flow cytometric analysis of central and peripheral lymphoid organs revealed that 8 week old transgenic mice were indistinguishable from littermate controls. Control mice with a non-mutated form of the c-MYC transgene succumbed rapidly to aggressive pro-B lymphomas, and reporter mice with a non-mutated EGFP in place of c-MYC confirmed lymphocyte-restricted expression of the transcriptional control elements used in the transgenes.

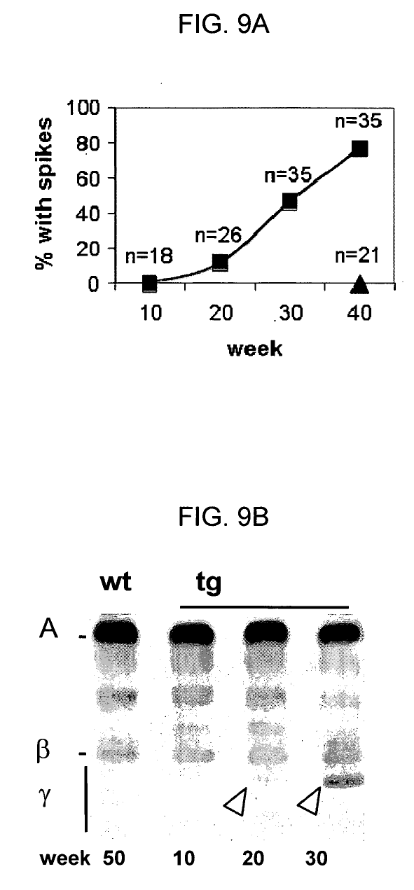

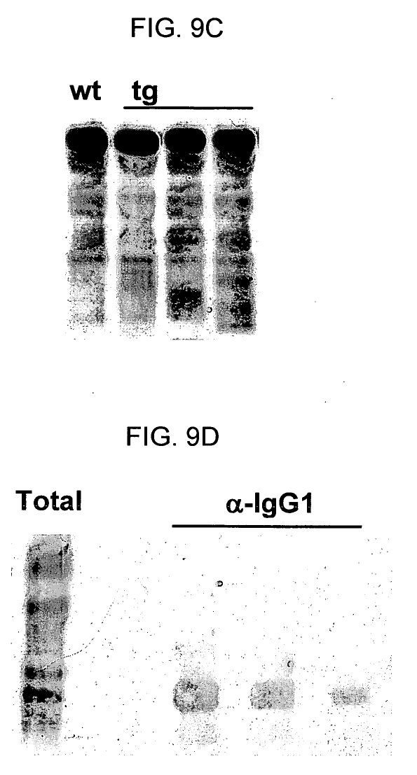

Similar to clinical diagnostic screening for the human plasma cell tumor, multiple myeloma, Vk*HAMYC mouse sera were collected and tested by protein electrophoresis. Spontaneous monoclonal spikes in the γ-fraction of serum could be detected at as early as 20 weeks of age (see FIG. 9A), were sustained, and mostly increased in intensity over time (see FIG. 9B). At 40 weeks, 5 out of 36 transgenic sera had two or more spikes (see FIG. 9C). Immunofixation for mouse isotypes was performed to determine the switched nature of the secreting cells, which were IgG1-switched in 6 out of 7 cases tested (see FIG. 9D).

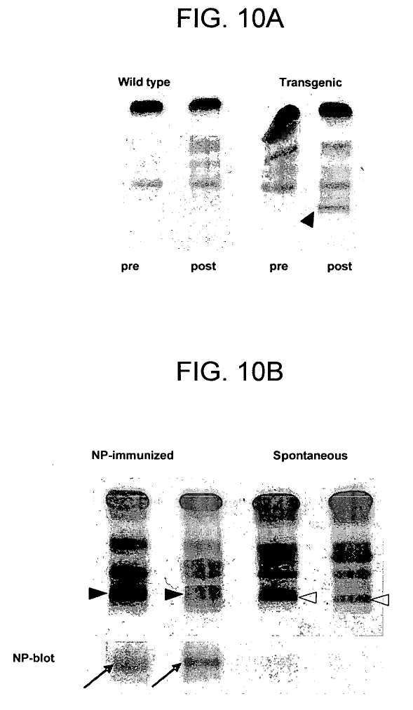

Representative mice (30-50 weeks of age with single spikes 1-3 fold greater intensity than the β-fraction of serum) were euthanized and subject to detailed analysis. No macroscopic abnormalities were observed at necropsy, including no evidence of lymphosplenomegaly. Flow cytometry revealed an increased number of B220 negative, CD138 positive plasma cells (range 4-8%; <1% in littermate controls) in the bone marrow, but not in the spleen of transgenic mice. CD138 positive plasma cells were staining for surface IgG1. Histologically, numerous peritrabecular bone marrow plasma cells were present. Plasma cells were positive for HA and human MYC indicating successful reversion of the stop codon. This result was confirmed by western blot with an anti-HA antibody. Plasma cells were also detected in peripheral blood smears of transgenics but not wild-type controls. Furthermore, mice were immunized with the model antigen NP-CGG. Two of eight transgenic mice developed gammopathies by 2 weeks after immunization (see FIG. 10A). These mice were sustained over time and responsive to the antigen (see FIG. 10B). Therefore, a large number of plasma cells reactive with a given antigen can be induced using the invention. Furthermore, such an increase in the proportion of plasma cells reactive with an immunized antigen can facilitate the generation of monoclonal antibodies to the antigen.

The results of this example demonstrate that an inactivated gene operatively linked to a hotspot for somatic hypermutation can be activated in vivo.

### EXAMPLE 2

This example further demonstrates the reactivation of an inactivated gene by placing the inactivated gene in a hotspot for somatic hypermutation.

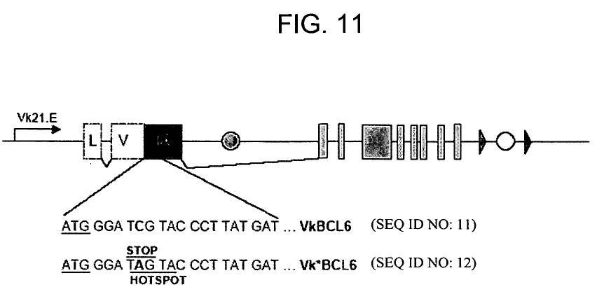

The BCL-6 gene is commonly involved in a form of germinal center cell lymphoma called diffuse large cell lymphoma. C57BL/6 mice were engineered to express a stop-mutated, HA-tagged (SEQ ID NO: 11 and SEQ ID NO: 12), human BCL-6 oncogene in B cells, under the control of mouse kappa light chain regulatory elements (Vk*BCL-6, FIG. 11). Promoter and enhancer elements were chosen and positioned because of their ability to drive SH, and the stop-mutation was overlapped with an rgyw hotspot. Two independent lines were generated. Control BCL-6 transgenes without a premature stop codon experienced B cell developmental arrest. Vk*BCL-6 transgenic mice developed splenic white plup lymphomas similar to human germinal center cell lymphomas.

The results of this example further demonstrate that an inactivated gene operatively linked to a hotspot for somatic hypermutation can be activated in vivo.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An isolated and purified nucleic acid comprising a gene functionally linked to a hotspot for somatic hypermutation, wherein the nucleic acid further comprises a nucleic acid sequence that inactivates or attenuates the gene.

2. The nucleic acid of claim 1, wherein the nucleic acid sequence inactivating or attenuating the gene is an upstream start codon, an upstream Kozak sequence, an upstream open reading frame, a stem-loop structure, a repressor binding sequence, an iron responsive element, a premature stop codon, a frame shift mutation, a mutant splice donor site, a mutant splice acceptor site, an internal ribosome entry site, a sequence that accelerates RNA degradation, or a sequence that encodes amino acids that accelerate protein degradation.

3. An isolated and purified nucleic acid comprising an inactivated or attenuated gene, wherein the gene is operatively linked to one or more immunoglobulin gene regulatory elements.

4. The nucleic acid of claim 3, wherein the immunoglobulin gene regulatory elements are kappa light chain gene regulatory elements.

5. An isolated and purified nucleic acid comprising an inactivated or attenuated gene, wherein the nucleic acid sequence inactivating the gene is within about one to about five nucleotides of a rgyw or a dgyw nucleic acid sequence.

6. The nucleic acid of claim 5, wherein the gene is operatively linked to immunoglobulin gene regulatory elements.

7. The nucleic acid of claim 1, wherein the gene is selected from the group consisting of c-MYC, BCL-1, BCL-2, BCL-3, BCL-6, N-MYC, L-MYC, v-MYC, MMSET, MAF, FGFR-3, MUM1/IRF-4, RAS, viral receptor genes, or site-specific recombinases.

8. The nucleic acid of claim 1, wherein the inactivated or attenuated gene is isolated from at least one other nucleic acid sequence found in the natural context of the gene.

9. An embryonic stem cell transformed with the nucleic acid of claim 1.

10. A method comprising transforming animal cells with the nucleic acid of claim 1.

11. A method comprising transforming cells of the hematopoietic lineage with the nucleic acid of claim 1.

12. A transgenic animal or progeny thereof whose germ cells and somatic cells comprise the nucleic acid of claim 1.

13. The animal of claim 12, wherein
(a) the animal is of a species that makes antibodies,
(b) the attenuated or inactivated gene is an oncogene, and
(c) the attenuated or inactivated gene is operatively linked to one or more immunoglobulin gene regulatory elements.

14. The animal of claim 13, wherein the attenuated or inactivated gene is a c-MYC oncogene.

15. An animal comprising the progeny of a cross between the animal of claim 13 and an animal deficient in the p16 tumor suppressor gene.

16. An isolated antibody produced by an animal of claim 13.

17. An isolated B cell of an animal of claim 13.

18. A hybridoma obtained from the fusion of a cell of claim 17 and a fusion partner.

19. An isolated antibody producing cell of an animal of claim 13.

20. A cell of claim 19, wherein the cell is adapted for growth in vitro.

21. A method of producing antibodies comprising culturing an isolated cell of claim 20 in a culture medium, and isolating the antibody from the culture medium.

22. A hybridoma obtained from the fusion of a cell of claim 19 and a fusion partner.

23. A transgenic blastocyst whose cells comprise the nucleic acid of claim 1.

24. A method of regulating the expression of a gene in a cell, the method comprising
(a) engineering a gene that is functionally inactive,
(b) cloning the functionally inactivated gene so as to be operatively linked to control elements that subject the gene to somatic hypermutation, and
(c) transforming cells with the inactivated gene functionally linked to somatic hypermutation control elements.

25. The method of claim 24, further comprising
(d) monitoring the transformed cells for expression of the gene.

26. The method of claim 24, wherein the gene produces RNA.

27. The method of claim 24, wherein the gene produces a protein.

28. The method of claim 24, wherein the functionally-inactivated gene produces a gene product without prior mutation, but the gene product does not produce a phenotypic effect on the cell prior to mutation.

29. An isolated monoclonal cell line expressing an antibody, wherein the cell line is obtained from an animal comprising the nucleic acid of claim 1.

30. A method comprising
(a) immunizing the animal of claim 13 with an antigen of interest,
(b) harvesting antibody-producing cells from the immunized animal, and
(c) isolating a cell producing antibody to the antigen of interest.

31. The method of claim 30, further comprising
(d) fusing the harvested antibody-producing cells from the animal with fusion partners.
